(19) United States
(12) Patent Application Publication
(10) Pub. No.: US 2017/0023668 A1
(43) Pub. Date: Jan. 26, 2017

(54) BEAMFORMING METHOD AND APPARATUS USING UNFOCUSED ULTRASONIC WAVES

(71) Applicant: ALPINION MEDICAL SYSTEMS CO., LTD., Hwaseong-si, Gyeonggi-do (KR)

(72) Inventors: Sun-yeob CHANG, Seoul (KR); Keonho SON, Seongnam-si (KR); Ja-woon KOO, Seoul (KR); Young-seop LIM, Seoul (KR)

(21) Appl. No.: 15/039,565

(22) PCT Filed: Nov. 29, 2013
(86) PCT No.: PCT/KR2013/010974

§ 371 Date: May 26, 2016

(30) Foreign Application Priority Data
Nov. 29, 2013 (KR) 10-2013-0146925

(51) Int. Cl. G01S 7/52; A61B 8/08; A61B 8/00; G01S 15/89; A61B 8/14

(52) U.S. Cl. CPC G01S 7/52085; G01S 15/8915; A61B 8/145; A61B 8/54; A61B 8/469; A61B 8/4483; A61B 8/5207

(57) ABSTRACT

A method and an apparatus for performing a beamforming by using unfocused ultrasounds are disclosed. Some embodiments of the present disclosure provide a method and an apparatus for performing a beamforming, when processing an ultrasound image obtained by transmitting unfocused ultrasounds to a field-of-view and receiving reflection signals from the field-of-view, which include calculating transmission delay times taken by ultrasounds transmitted by transmit elements of a transducer to arrive at a receive focusing point, calculating reception delay times taken by reflection ultrasound signals reflected at the receive focusing point to arrive at receive elements of the transducer, and applying the calculations of the transmission delay times and the reception delay times to reception signals at the receive elements.

### TECHNICAL FIELD

The present disclosure relates to a method and an apparatus for performing a beamforming by using unfocused ultrasounds.

### BACKGROUND

The statements in this section merely provide background information related to the present disclosure and do not necessarily constitute prior art.

An ultrasound system transmits an ultrasound to a subject by using a probe, receives a reflected signal from the subject, and generates an ultrasound image by converting the reflected signal into an electrical signal. The ultrasound system has noninvasive and nondestructive property, and hence it is widely used in a medical field for obtaining biometric data. Configured to provide an image of an internal tissue of a body in real time instead of a surgery for incising and observing the body, the ultrasound system is generally used in the medical field.

In recent years, an image processing technology for achieving high-speed image processing has become influential, in which a plane wave is transmitted to a subject, a reflected signal corresponding to the plane wave is received from the subject, and an image frame is processed at high speed based on the reflected signal. Utilizing the plane wave for the high-speed image processing, however, has an issue of increasing the frame rate of an ultrasound image generated thereby while degrading the image quality more or less than when a focused ultrasound is used.

### DISCLOSURE

### Technical Problem

It is an object of some embodiments of the present disclosure to provide a method and an apparatus for performing a beamforming, when processing an ultrasound image obtained by transmitting unfocused ultrasounds to a field-of-view and receiving reflection signals from the field-of-view, which include calculating transmission delay times taken by ultrasounds transmitted by transmit elements of a transducer to arrive at a receive focusing point, calculating reception delay times taken by reflection ultrasound signals reflected at the receive focusing point to arrive at receive elements of the transducer, and applying the calculations of the transmission delay times and the reception delay times to reception signals at the receive elements.

### SUMMARY

According to some embodiments of the present disclosure, a method for performing a beamforming by an ultrasound medical apparatus includes transmitting, by a transducer, an unfocused ultrasound to a field-of-view, calculating a transmission delay time of a transmission path through which an ultrasound is transmitted by one of a plurality of transmit elements of the transducer and arrives at a receive focusing point and calculating reception delay times of reception paths through which the ultrasound reflected at the receive focusing point arrives at a plurality of receive elements, respectively, generating a plurality of delay signals for respective reception signals of the receive elements by applying the calculating of the transmission delay time and the reception delay times to remaining transmit elements of the plurality of transmit elements, and performing the beamforming by summing the plurality of delay signals.

According to some embodiments of the present disclosure, an ultrasound medical apparatus includes a transducer and a beamformer. The transducer is configured to transmit unfocused ultrasounds to a field-of-view. And the beamformer is configured to calculate a transmission delay time of a transmission path through which an ultrasound is transmitted by one of a plurality of transmit elements of the transducer and arrives at a receive focusing point and calculate reception delay times of reception paths through which the ultrasound reflected at the receive focusing point arrives at a plurality of receive elements, respectively, to generate a plurality of delay signals for respective reception signals of the receive elements by applying calculations of the transmission delay time and the reception delay times to remaining transmit elements of the plurality of transmit elements, respectively, and to perform a beamforming by summing the plurality of delay signals.

### Advantageous Effects

As described above, according to some embodiments of the present disclosure, when processing an ultrasound image obtained by transmitting an unfocused ultrasound to a field-of-view and receiving a reflected signal from the field-of-view, a signal can be delayed by applying a transmission delay time required for an ultrasound transmitted by a transmit element of a transducer to arrive at a receive focusing point and a reception delay time for the reflected signal reflected at the receive focusing point to arrive at a receive element of the transducer on a signal received by the receive element.

Further, according to some embodiments of the present disclosure, when processing an ultrasound image obtained by transmitting an unfocused ultrasound to a field-of-view and receiving a reflected signal from the field-of-view, the ultrasound image can be generated considering time delays of transmission and reception paths of all reflected signals corresponding to an ultrasound transmitted by at least one transmit element of the transducer. Moreover, according to some embodiments of the present disclosure, as the ultrasound image is generated considering all the reflected signals, signal-to-noise ratio, contrast, and resolution can be improved compared to an ultrasound image generated by a typical plane wave.

Further, according to some embodiments of the present disclosure, as the image processing can be performed with a single frame, the frame rate of the ultrasound image is not degraded, and no moving artifact is caused by a movement of the subject. Moreover, as there is no artifact caused by a movement of the subject, the embodiments of the present disclosure can be applied to various image modes including a color flow mode and a Doppler mode. In addition, when storing raw data on which no beamforming is performed in a storage unit, a size of the stored data can be reduced.

### BRIEF DESCRIPTION OF DRAWINGS

FIG. 1 is a block diagram of an ultrasound medical apparatus according to some embodiments of the present disclosure.

FIG. 2 is a schematic diagram for illustrating a dynamic focusing according to some embodiments of the present disclosure.

FIGS. 3A and 3B are schematic diagrams for illustrating a receive dynamic focusing and a transmit and receive dynamic focusing according to some embodiments of the present disclosure.

FIGS. 4A and 4B are schematic diagrams for illustrating a beamforming process according to some embodiments of the present disclosure.

FIGS. 5A to 5D are schematic diagrams for illustrating a beamforming in a process of receiving a reflected signal according to some embodiments of the present disclosure.

FIG. 6 is a flowchart of a process procedure for a method for performing a beamforming by using an unfocused ultrasound according to some embodiments of the present disclosure.

FIG. 7 is a schematic diagram for illustrating various beamforming processes according to some embodiments of the present disclosure.

### DETAILED DESCRIPTION

Exemplary embodiments of the present disclosure are described in detail below with reference to the accompanying drawings.

An ultrasound medical apparatus 100 has a transducer 110 including a plurality of elements, of which an element that transmits an unfocused ultrasound to a field-of-view (FOV) is referred to as a “transmit element”. An element that receives a reflected signal from a receive focusing point in the field-of-view, among the elements of the transducer 110, is referred to as a “receive element”. A path through which the unfocused ultrasound is transmitted from the transmit element to the receive focusing point in the field-of-view is defined as a “transmission path”, and a path through which the reflected signal travels from the receive focusing point in the field-of-view to the receive element is referred to as a “reception path”. The receive focusing point can be selected by a user instruction, and the number of transmit elements is not necessarily the same as that of receive elements.

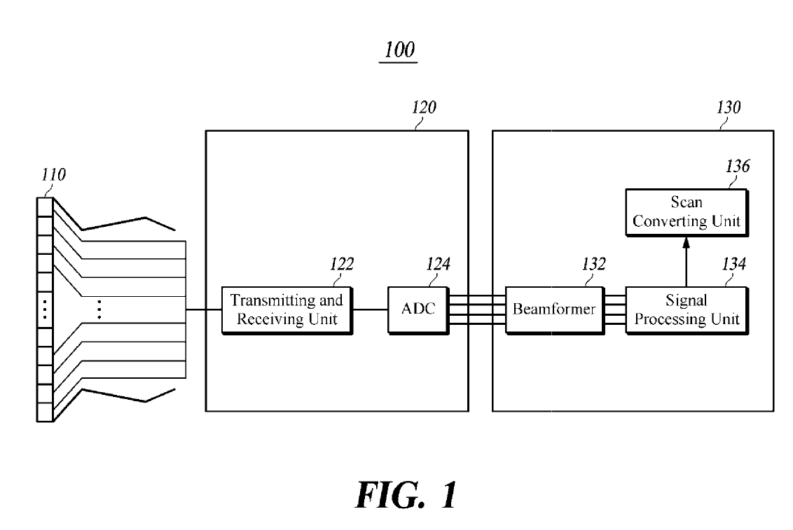

FIG. 1 is a block diagram of the ultrasound medical apparatus 100 according to some embodiments of the present disclosure.

The ultrasound medical apparatus 100 according to some embodiments of the present disclosure performs a beamforming based on software, and includes the transducer 110, a front-end processing unit 120 and a host device 130. The constituent elements of the ultrasound medical apparatus 100 are not limited to the above-mentioned elements.

The front-end processing unit 120 includes a transmission and reception unit 122 and an analog-to-digital converter 124. The host device 130 includes a beamformer 132, a signal processing unit 134 and a scan converting unit 136. The host device 130 performs a software-based parallel processing for achieving high-speed image processing, and its architecture includes a multicore central processing unit (CPU) and a graphic processing unit (GPU) performing a parallel processing in a plurality (e.g., several thousands) of processors in a simultaneous manner.

The front-end processing unit 120 and the host device 130 can be connected to each other with a full parallel path to perform the software-based high-speed image processing by using, e.g., a peripheral component interconnect (PCI) Express interface.

As the ultrasound medical apparatus 100 according to some embodiments of the present disclosure performs the software-based high-speed image processing, the full parallel path connection structure between the front-end processing unit 120 and the host device 130 facilitates the processing of the ultrasound image at high speed. When a user desires to view the ultrasound image processed at high speed depending on a type of the subject in the field-of-view or serving the purpose of diagnosis, the ultrasound medical apparatus 100 can provide the ultrasound image generated based on the unfocused ultrasound in a short time.

After transmitting the unfocused ultrasound to the field-of-view, the transducer 110 receives a reflected signal from the receive focusing point corresponding to the unfocused ultrasound. The unfocused ultrasound includes at least one of a plane wave or a wide beam. The reflected signal corresponding to the plane wave can be processed with the software-based high-speed image processing. The transducer 110 can transmit unfocused ultrasounds having different frequencies to the field-of-view under a control of the beamformer 132 (or a separate control unit). In some embodiments, the transducer 110 is implemented as a transducer array, and is configured to transmit the unfocused ultrasound to the field-of-view by using a transducer element in the transducer array and to receive the reflected signal reflected at the field-of-view. Further, the transducer 110 is configured to transmit a focused ultrasound to a focusing area under a control of the transmission and reception unit 122, and to receive a reflected signal from the focusing area corresponding to the focused ultrasound.

A configuration of the front-end processing unit 120 is described below.

The transmission and reception unit 122 applies a voltage pulse to the transducer 110 to allow each transducer element of the transducer 110 to output a focused ultrasound or an unfocused ultrasound. The transmission and reception unit 122 performs a function of switching between transmission and reception to allow the transducer 110 to perform the transmission and the reception in an alternate manner. The analog-to-digital converter 124 converts the analog reflected signal received from the transmission and reception unit 122 into a digital signal, and sends the digital signal to the beamformer 132.

A configuration of the host device 130 is described below.

The beamformer 132 generates a delay time required to transmit the unfocused ultrasound to the field-of-view when processing an ultrasound image by using the unfocused ultrasound. In other words, the beamformer 132 applies the same delay time (e.g., zero) to each of the elements to allow the unfocused ultrasound to be transmitted to the front, or applies a predetermined delay time to each of the elements to allow the unfocused ultrasound to be transmitted in a direction other than the front.

The beamformer 132 provides a time delay for focusing the reflected signal received from the transducer 110, and adjusts a dynamic focusing of the reflected signal. The beamformer 132 generates a receive focusing signal by summing electrical digital signals outputted from the analog-to-digital converter 124 to form a beam. The beamformer 132 combines the digitalized signals into a single signal. At this time, reflected signals having the same phase are combined at the beamformer 132, applied with various signal processing schemes by the signal processing unit 134, and then outputted to a display unit via the scan converting unit 136. The beamformer 132 performs the dynamic focusing by applying different amounts of delay (determined based on a receive focusing position) to the signals received from the analog-to-digital converter 124 and combining delayed signals. For example, the beamformer 132 combines the reflected signals received from the transducer elements into a single signal for a subsequent signal processing. The beamformer 132 generates a combined signal that is obtained by combining the reflected signals received from all the transmit elements in order to make a single reflected signal for each receive focusing point in the field-of-view. The combined signal generated in the above manner is sent to the signal processing unit 134 by the beamformer 132, and is eventually sent to the display unit that changes the combined signal to a digital format to store image data.

An operation of the beamformer 132 is described below.

The beamformer 132 generates delay signals for delaying signals by applying a transmission delay time τⱼ₍ₜₓ₎ and a reception delay time τᵢ₍ᵣₓ₎ to signals received by receive elements Rxᵢ (where i=1 to N, N being an integer equal to or larger than 2). Here, the transmission delay time τⱼ₍ₜₓ₎ is the time required for ultrasounds transmitted from a plurality of transmit elements Txⱼ (where j=1 to M, and M is an integer equal to or larger than 2) selected from the transmit elements of the transducer 110 to arrive at the receive focusing point in the field-of-view, and the reception delay time τᵢ₍ᵣₓ₎ is the time required for signals from the receive focusing point to arrive at the receive elements (Rxᵢ). The beamformer 132 performs the beamforming based on N×M delay signals generated from the above-mentioned operation of generating the delay signals. The beamformer 132 performs the beamforming by generating the N×M delay signals by performing the above-mentioned process of generating the delay signals for each of the M transmit elements and the N receive elements.

In some embodiments, the plurality of transmit elements refers to such transducer elements selectively determined as having transmitted the unfocused ultrasounds that are estimated to have arrived at the receive focusing point, and the plurality of transmit elements determined may change according to the receive focusing point.

For example, the plurality of transmit elements selected from the transmit elements of the transducer 110 is determined based on a focal depth of the receive focusing point. Typically, when determining the number of transmit elements at the time of transmitting an ultrasound, the number of transmit elements is adjusted based on a user instruction (focal depth and F number, i.e. ratio of aperture size depending on depth) inputted from the user input unit. However, in some embodiments, the beamformer 132 selectively determines a plurality of transmit elements which had transmitted unfocused ultrasounds that are estimated to have arrived at the receive focusing point based on the focal depth of the receive focusing point and the F number according to the determined position of the receive focusing point in the field-of-view. Although the beamformer 132 is described as having a function of determining the number of the plurality of transmit elements to be selected from the transmit elements of the transducer 110, this function can also be achieved by providing a separate control unit.

The number of the plurality of transmit elements selected from the transmit elements of the transducer 110 increases in proportion to the focal depth of the receive focusing point. In some embodiments, the receive focusing point is a position selected in a region-of-interest (POI). When the image is in a multi-gate Doppler mode, the receive focusing point may be a gate position.

A transmit apodization can be achieved in the process of performing the beamforming. The beamformer 132 determines a first weight (W₁ⱼ) based on the receive focusing point and positions of the plurality of transmit elements (Txⱼ) selected from the transmit elements of the transducer 110. The beamformer 132 generates weighted delay signals by applying the first weight (W₁ⱼ) to the delay signals on which the transmission delay time τⱼ₍ₜₓ₎ has been applied. The beamformer 132 performs the beamforming by weighted summing N×M weighted delay signals generated by the process of generating the weighted delay signals.

Further, a receive apodization can be achieved in the process of performing the beamforming. In its process of applying a receive weight, the beamformer 132 determines a second weight (W₂ᵢ) based on the receive focusing point and positions of the receive elements (Rxᵢ). The beamformer 132 generates weighted delay signals by applying the second weight (W₂ⱼ) to the delay signals on which the reception delay time τᵢ₍ₜₓ₎ has been applied. The beamformer 132 performs the beamforming by weighted summing N×M weighted delay signals generated by the process of generating the weighted delay signals.

Moreover, both the transmit apodization and the receive apodization can be achieved in the process of performing the beamforming. The beamformer 132 determines a first weight (W₁ⱼ) based on the receive focusing point and positions of the plurality of transmit elements (Txⱼ) selected from the transmit elements of the transducer 110. The beamformer 132 determines a second weight (W₂ⱼ) based on the receive focusing point and positions of the receive elements (Rxᵢ). The beamformer 132 generates weighted delay signals by applying the first weight (W₁ⱼ) and the second weight (W₂ⱼ) to the delay signals on which the transmission delay time τⱼ₍ₜₓ₎ and the reception delay time τᵢ₍ₜₓ₎ have been applied. The beamformer 132 performs the beamforming by weighted summing N×M weighted delay signals generated by the process of generating the weighted delay signals.

The above-mentioned unfocused ultrasound beamforming process can also be performed in an image reconstruction mode for reconstructing an image by using pre-stored data, such as a cine mode and a virtual rescan mode. More specifically, the beamformer 132 operates to store a reflected signal (data) reflected at the receive focusing point corresponding to the unfocused ultrasound in a separate storage unit. Thereafter, when an image reconstruction mode is selected by the user instruction, the beamformer 132 performs the above-mentioned processes of generating the delay signal and performing the beamforming by using the reflected signal corresponding to the unfocused ultrasound stored in the storage unit. For example, the beamformer 132 stores the reflected signal (data) reflected at the receive focusing point corresponding to the unfocused ultrasound in a separate storage unit, and when reconstructing an image in an image mode such as the cine mode or the virtual rescan mode, performs the above-mentioned processes of generating the above-mentioned delay signal and performing the beamforming by using the previously stored data.

An operation of the beamformer 132 according to some embodiments of the present disclosure is described below.

The beamformer 132 calculates a transmission delay time caused by a transmission path through which an ultrasound transmitted from any one transmit element among the transmit elements of the transducer 110 arrives at the receive focusing point, and calculates a reception delay time caused by a reception path through which the reflected signal reflected at the receive focusing point arrives at each of the receive elements. The beamformer 132 generates a plurality of delay signals for the respective reception signals of the receive elements by applying the process of calculating the transmission delay time and the reception delay time to the rest of the transmit elements, and performs the beamforming by summing the generated plurality of delay signals. In some embodiments, the beamformer 132 does not impose upon all the respective transmit elements the beamforming performed by applying the delay times T depending on the transmission and reception paths to the respective receive elements' receive signals but impose such beamforming upon some of all of the transmit elements.

Depending on the depth of the receive focusing point in the field-of-view, the beamformer 132 performs the beamforming by applying a transmission weight corresponding to the transmission path and applying a reception weight corresponding to the reception path.

In response to the ultrasound transmitted by every single element among a plurality of selected transmit elements from the elements of the transducer 110, the beamformer 132 generates reflection signals reflecting at a receive focusing point with concurrently defined time delays applied, and then combines the generated reflection signals into a combined signal. The beamformer 132 then combines a set of combined signals corresponding respectively to the selected transmit elements of the transducer 110 with the concurrently defined time delays applied, in order to generate a reception signal.

The beamformer 132 performs the beamforming by summing a transmission time delay τⱼ₍ₜₓ₎ and a reception time delay τᵢ₍ᵣₓ₎, wherein the transmission time delay τⱼ₍ₜₓ₎ is the time for a J-th unfocused ultrasound transmitted from a J-th element among the selected transmit elements of the transducer 110 to arrive at the receive focusing point, and the reception time delay τᵢ₍ᵣₓ₎ is the time for a j-th reflected signal corresponding to the j-th unfocused ultrasound to arrive at an i-th element. In the beamforming operation, the beamformer 132 applies a transmission weight to a transmission path through which the j-th ultrasound arrives at the receive focusing point depending on the depth of the receive focusing point in the field-of-view, and it applies a reception weight to a reception path through which the j-th reflected signal arrives at the i-th element among the receive elements. The beamformer 132 performs the beamforming on frequency compound frame data when it is generated.

The beamformer 132 allows the frame data to be generated based on the reception signal generated by performing the beamforming for each of the elements selected from the transmit elements of the transducer 110. The beamformer 132 generates at least one frame data based on reflected signals corresponding to unfocused ultrasounds having different frequencies from each other before performing the beamforming, and generates the frequency compound frame data by frequency compounding at least one frame data.

The signal processing unit 134 obtains data for a single scanline by converting the reflected signal of a reception scanline focused by the beamformer 132 into a baseband signal and detecting an envelope by using a quadrature demodulator. The signal processing unit 134 processes the data generated by the beamformer 132 as digital signals.

The scan converting unit 136 records the data obtained by the signal processing unit 134 in a memory, aligns a scan direction of the data with a pixel direction of a display unit (e.g., a monitor), and maps the relevant data to pixel positions of the display unit. The scan converting unit 136 converts a format of the ultrasound image data into a data format used in the display unit in a scanline display format.

In some embodiments, the ultrasound medical apparatus 100 further includes a user input unit that receives an instruction from an operation or an input of a user. In some embodiments, the user instruction includes a setting instruction for controlling the ultrasound medical apparatus 100. In some embodiments, the ultrasound medical apparatus 100 further includes a storage unit that stores therein a reflected signal (a pre-receive-beamforming signal) having passed through the analog-to-digital converter 124 or a reflected signal on which the receive beamforming is performed (a post-receive-beamforming signal).

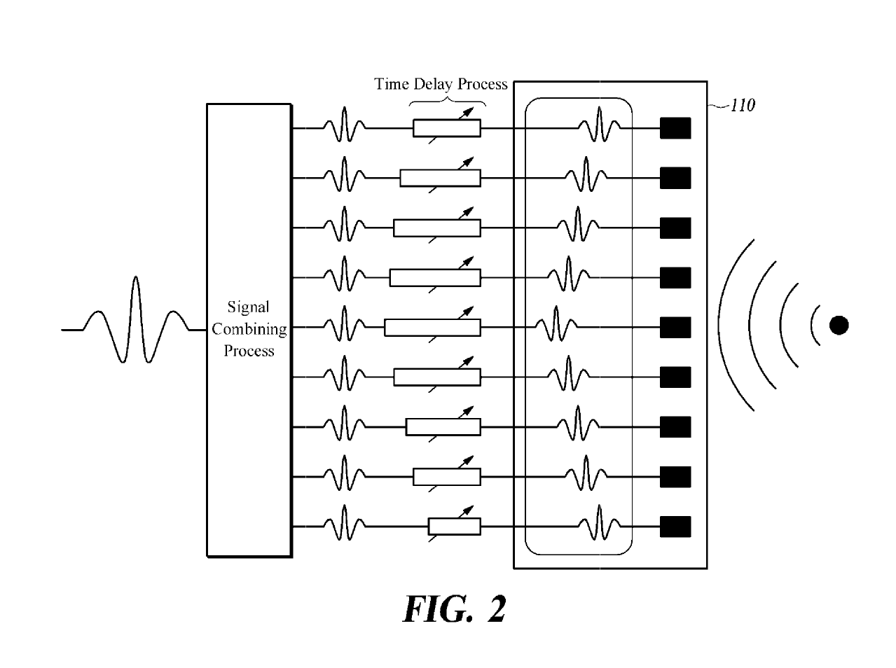

FIG. 2 is a schematic diagram for illustrating a dynamic focusing according to some embodiments of the present disclosure.

A dynamic receive focus is described with reference to FIG. 2. When the transducer 110 of the ultrasound medical apparatus 100 is a transducer array, the beamformer 132 focuses the reflected signal received from the receive focusing position in the field-of-view. After transmitting the ultrasound, the ultrasound medical apparatus 100 receives the reflected signal from the receive focusing point in the field-of-view by using a group of oscillators (receive elements) of the transducer 110. The reflected signals are amplified as they reach the group of oscillators (receive elements), and they are combined at the beamformer 132 that generates a single signal from each receive focusing point. However, in this case, a slight compensation for a time difference is needed due to a difference in a distance (e.g., as shown in FIG. 2) through which a return pulse travels before the reflected signals are combined. If the reflected signals reflected at the receive focusing point in the field-of-view were directly combined without being temporally tuned, the signals are partially canceled out. For example, a positive duration of a signal from an oscillator (any one element among the receive elements) occurs at a time during which a signal from another oscillator (another element among the receive elements) is negative, these two signals are canceled out when being combined.

Compensating for the time difference can be achieved by applying a delay signal that is set based on the depth of the receive focusing point in the field-of-view. FIG. 2 shows the reflected signals tuned to be in-phase after they underwent the time delay process. The required time delay depends on the depth of the receive focusing point.

Unlike a transmission from an array-type oscillator of the transducer 110, the reflected signals during the reception undergo a dynamic focusing. When an acoustic pulse is transmitted, a focal length of the reception at the array-type oscillator is determined in a cursory manner at first. As the transmission pulse increases and depending on time after the reflected signal returns from the receive focusing point deeper inside the field-of-view, the receive focus automatically shifts by following or tracking a position where an acoustic pulse hits the receive focusing point deep inside the field-of-view. The tracking for achieving the dynamic receive focus is a rapid progress performed at all positions being within a time limit required for the reflected signal to return from all depths. Using the dynamic focus enables a single oscillator transducer to expand the depth of focus much more than using a single focal length in an array. A transmission focal length can be selected by the user in a transducer array. The dynamic receive focus can be applied to all depths of the receive focusing point in the field-of-view.

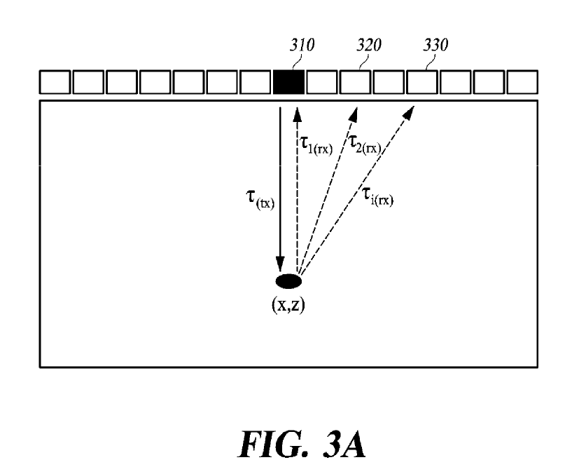

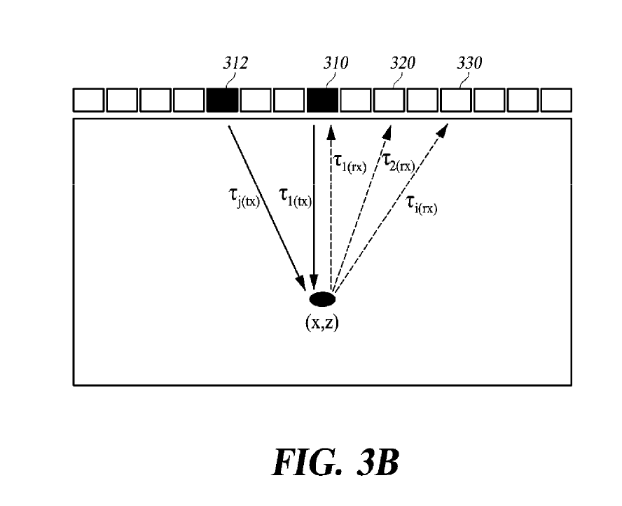

FIGS. 3A and 3B are schematic diagrams for illustrating a receive dynamic focusing and a transmit and receive dynamic focusing according to some embodiments of the present disclosure.

A receive dynamic focusing by using an unfocused ultrasound is described below with reference to FIG. 3A. In FIG. 3A, with respect to a single element 310 among a plurality of selected transmit elements 310, 320, 330 from the elements of the transducer 110, the ultrasound medical apparatus 100 performs a beamforming taking account of time delays corresponding to the paths through which an ultrasound is transmitted from the element 310, reflected at a receive focusing point (x, z) in the field-of-view and arrives at respective receive elements 310, 320 and 330, by applying the time delays to reception signals at the respective receive elements.

In FIG. 3A, a time required for the ultrasound transmitted from the element 310 among the plurality of the selected transmit elements of the transducer 110 to reach the receive focusing point (x, z) in the field-of-view is defined as a transmission delay time Td. A time delay caused by a path through which the ultrasound is reflected at the receive focusing point (x, z) and arrives at the element 310 among the receive elements is defined as τ₁₍ᵣₓ₎, a time delay caused by a path through which the ultrasound is reflected at the receive focusing point (x, z) and arrives at the element 320 among the receive elements is defined as τ₂₍ᵣₓ₎, and a time delay caused by a path through which the ultrasound is reflected at the receive focusing point (x, z) and arrives at the element 330 among the receive elements is defined as τᵢ₍ᵣₓ₎.

A delay time for the element 310 among the receive elements of the transducer 110 is equal to τ₍ₜₓ₎+τ₁₍ᵣₓ₎, a time delay for the element 320 among the receive elements of the transducer 110 is equal to τ₍ₜₓ₎+τ₂₍ᵣₓ₎. When the element 330 among the receive elements of the transducer 110 is an i-th element, a delay time for the i-th element 330 is equal to τ₍ₜₓ₎+τᵢ₍ᵣₓ₎, and a beamforming signal is defined by Equation 1.

Rᵢ(t−(τ₍ₜₓ₎+τᵢ₍ᵣₓ₎) Equation 1

where Rᵢ is a signal received by i-th element 330, τ₍ₜₓ₎ is a time required for the ultrasound transmitted from the element 310 to arrive at the receive focusing point, and τᵢ₍ᵣₓ₎ is a time required for the reflected signal to arrive at the i-th element 330 from the receive focusing point.

When using Equation 1, the ultrasound medical apparatus 100 according to some embodiments of the present disclosure can apply a receive weight for the i-th element 330 as Equation 2.

$${\sum }_{i} {W}_{i} {R}_{i} ( t - ( {τ}_{( tx )} + {τ}_{i ( rx )} ) \qquad Equation 2$$

where Wᵢ is a receive weight for the i-th element 330, Rᵢ is a signal received by the i-th element 330, τ₍ₜₓ₎ is a time required for the ultrasound transmitted from the element 310 to arrive at the receive focusing point, and τᵢ₍ᵣₓ₎ is a time required for the reflected signal to arrive at the i-th element 330 from the receive focusing point.

A transmission and reception dynamic focusing by using an unfocused ultrasound is described below with reference to FIG. 3B. In FIG. 3B, with respect to a plurality of elements 310 and 312 selected from the elements 310, 320, 330 in the transducer 110, the ultrasound medical apparatus 100 performs a beamforming taking account of time delays corresponding to the paths through which an ultrasound is transmitted from the plurality of selected elements 310 and 312, reflected at a receive focusing point (x, z) in the field-of-view and arrives at the respective receive elements 310, 320 and 330, by applying the time delays to reception signals at the respective receive elements.

In FIG. 3B, a time, which is required for the ultrasound transmitted from the element 310 among the plurality of elements selected from the transmit elements of the transducer 110 to arrive at the receive focusing point (x, z) in the field-of-view, is defined as a transmission delay time τ₁₍ₜₓ₎, and a time, which is required for the ultrasound transmitted from the element 312 among the selected transmit elements of the transducer 110 to arrive at the receive focusing point (x, z) in the field-of-view, is defined as a transmission delay time τⱼ₍ₜₓ₎.

A time delay, which is caused by a path through which the ultrasound is reflected at the receive focusing point (x, z) and arrives at the element 310 among the receive elements, is defined as τ₁₍ᵣₓ₎. A time delay, which is caused by a path through which the ultrasound is reflected at the receive focusing point (x, z) and arrives at an element 320 among the receive elements, is defined as τ₂₍ᵣₓ₎. And a time delay, which is caused by a path through which the ultrasound is reflected at the receive focusing point (x, z) and arrives at an element 330 among the receive elements, is defined as τᵢ₍ᵣₓ₎.

A delay time for the reflected signal corresponding to the ultrasound transmitted from the element 310 is equal to τ₁₍ₜₓ₎+τ₁₍ᵣₓ₎ at the element 310, and a delay time for the reflected signal corresponding to the ultrasound transmitted from the element 312 is equal to τⱼ₍ₜₓ₎+τ₁₍ᵣₓ₎.

At the element 320 among the receive elements of the transducer 110, a delay time for the reflected signal corresponding to the ultrasound transmitted from the transmit element 310 is equal to τ₁₍ₜₓ₎+τ₂₍ᵣₓ₎, and a delay time for the reflected signal corresponding to the ultrasound transmitted from the transmit element 312 is equal to τⱼ₍ₜₓ₎+τ₂₍ᵣₓ₎.

At the element 330 among the receive elements of the transducer 110, a delay time for the reflected signal corresponding to the ultrasound transmitted from the transmit element 310 is equal to τ₁₍ₜₓ₎+τᵢ₍ᵣₓ₎, and a delay time for the reflected signal corresponding to the ultrasound transmitted from the transmit element 312 is equal to τⱼ₍ₜₓ₎+τᵢ₍ᵣₓ₎ with the resultant beamforming signal defined as Equation 3.

Rᵢ(t−(τⱼ₍ₜₓ₎+τᵢ₍ᵣₓ₎)) Equation 3

where Rᵢ is a signal received by the i-th element 330, τⱼ₍ₜₓ₎ is a time required for the ultrasound transmitted from the j-th element 312 to arrive at the receive focusing point, and τᵢ₍ᵣₓ₎ is a time required for the reflected signal to arrive at the i-th element 330 from the receive focusing point.

When using Equation 3, the ultrasound medical apparatus 100 according to some embodiments of the present disclosure can apply transmission and reception weights as Equation 4.

$${\sum }_{j} {\sum }_{i} {W}_{ij} {R}_{i} ( t - ( {τ}_{j ( tx )} + {τ}_{i ( rx )} ) ) \qquad Equation 4$$

where Wᵢ is a transmission weight for the ultrasound transmitted from the j-th element 312 and a reception weight for the i-th element 330, Rᵢ is a signal received by the i-th element 330, τⱼ₍ₜₓ₎ is a time required for the ultrasound transmitted from the j-th element 312 to arrive at the receive focusing point, and τᵢ₍ᵣₓ₎ is a time required for the reflected signal to arrive at the i-th element 330 from the receive focusing point.

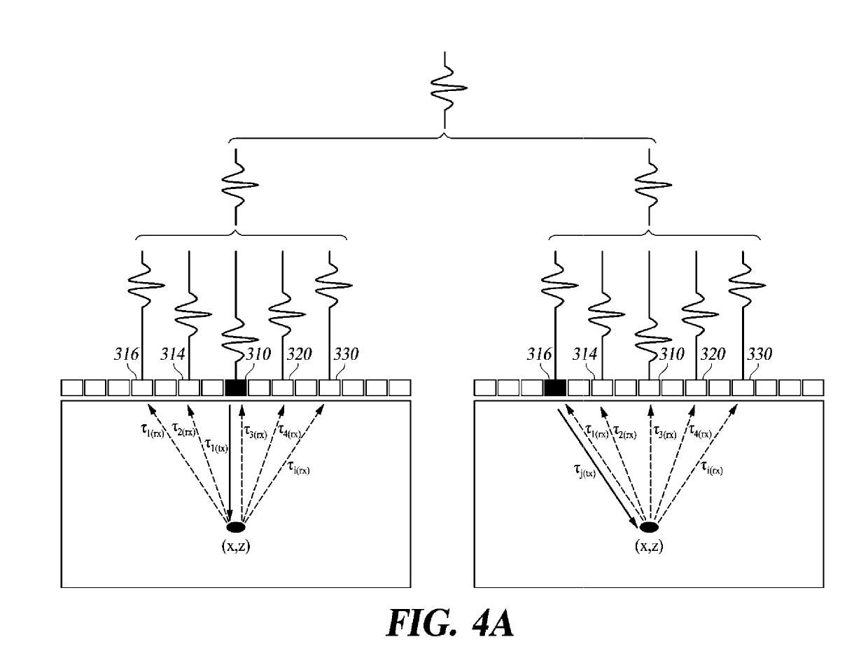

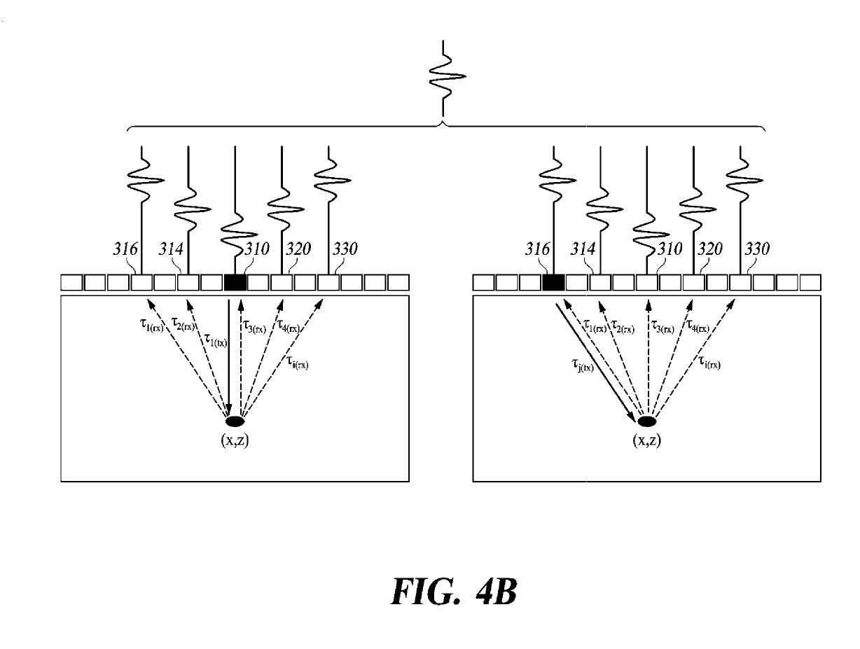

FIGS. 4A and 4B are schematic diagrams for illustrating a beamforming process according to some embodiments of the present disclosure.

A beamforming at the time of transmitting an ultrasound at the element 310 according to some embodiments of the present disclosure is described with reference to FIG. 4A.

For respective ones of a plurality of selected transmit elements from the elements of the transducer 110, the ultrasound medical apparatus 100 performs the beamforming taking account of time delays corresponding to transmission and reception paths through which an ultrasound is transmitted from the single element 310, reflected at a receive focusing point (x, z) in the field-of-view and arrives at respective receive elements 310, 314, 316, 320 and 330, by applying the time delays to reception signals at the respective receive elements.

In FIG. 4A, a time required for the ultrasound transmitted from the element 310 among the selected transmit elements of the transducer 110 to arrive at the receive focusing point (x, z) in the field-of-view is defined as a transmission delay time τ₁₍ₜₓ₎.

Further, a time delay caused by a path through which the ultrasound transmitted from the element 310 is reflected at the receive focusing point (x, z) and arrives at the element 316 among the receive elements is defined as τ₁₍ᵣₓ₎ a time delay caused by a path through which the ultrasound is reflected at the receive focusing point (x, z) and arrives at the element 314 among the receive elements is defined as τ₂₍ᵣₓ₎, a time delay caused by a path through which the ultrasound is reflected at the receive focusing point (x, z) and arrives at the element 310 among the receive elements is defined as τ₃₍ᵣₓ₎, a time delay caused by a path through which the ultrasound is reflected at the receive focusing point (x, z) and arrives at the element 320 among the receive elements is defined as τ₄₍ᵣₓ₎, and a time delay caused by a path through which the ultrasound is reflected at the receive focusing point (x, z) and arrives at the element 330 among the receive elements is defined as τᵢ₍ᵣₓ₎.

A delay time for the element 316 among the receive elements of the transducer 110 is equal to τ₍ₜₓ₎+τd, and a time delay for the element 314 among the receive elements of the transducer 110 is equal to τ₍ₜₓ₎+τ₂₍ᵣₓ₎. A time delay for the element 310 among the receive elements of the transducer 110 is equal to τ₍ₜₓ₎+τ₃₍ᵣₓ₎, a time delay for the element 320 among the receive elements of the transducer 110 is equal to τ₍ₜₓ₎+τ₄₍ᵣₓ₎. Further, when the element 330 among the receive elements of the transducer 110 is an i-th element, a time delay for the element 330 is equal to τ₍ₜₓ₎+τᵢ₍ᵣₓ₎.

Thereafter, in response to the ultrasound transmitted by the single element 310 among a plurality of selected transmit elements from the elements of the transducer 110, the ultrasound medical apparatus 100 generates reception signals with time delays applied, which are defined concurrently corresponding to the paths through which the transmitted ultrasound is reflected at a receive focusing point (x, z) and arrives at the receive elements 310, 314, 316, 320, 330, and then combines the generated reception signals into a first combined signal.

A beamforming at the time of transmitting an ultrasound at the element 316 according to some embodiments of the present disclosure is described below.

For the respective ones of the plurality of selected transmit elements from the elements of the transducer 110, the ultrasound medical apparatus 100 performs the beamforming taking account of time delays corresponding to the paths through which an ultrasound is transmitted from the single element 316, reflected at the receive focusing point (x, z) in the field-of-view and arrives at respective receive elements 310, 314, 316, 320 and 330, by applying the time delays to reception signals at the respective receive elements.

In FIG. 4A, a time required for the ultrasound transmitted from the element 316 among the selected transmit elements of the transducer 110 to arrive at the receive focusing point (x, z) in the field-of-view is defined as a transmission delay time τⱼ₍ₜₓ₎.

Further, a time delay caused by a path through which the ultrasound transmitted from the element 316 is reflected at the receive focusing point (x, z) and arrives at the element 316 among the receive elements is defined as τ₁₍ᵣₓ₎, a time delay caused by a path through which the ultrasound is reflected at the receive focusing point (x, z) and arrives at the element (314) among the receive elements is defined as τ₂₍ᵣₓ₎, a time delay caused by a path through which the ultrasound is reflected at the receive focusing point (x, z) and arrives at the element 310 among the receive elements is defined as τ₃₍ᵣₓ₎, a time delay caused by a path through which the ultrasound is reflected at the receive focusing point (x, z) and arrives at the element 320 among the receive elements is defined as τ₄₍ᵣₓ₎, and a time delay caused by a path through which the ultrasound is reflected at the receive focusing point (x, z) and arrives at the element 330 among the receive elements is defined as τᵢ₍ᵣₓ₎.

A delay time for the element 316 among the receive elements of the transducer 110 is equal to τⱼ₍ₜₓ₎+τ₁₍ᵣₓ₎, and a delay time for the element 314 among the receive elements of the transducer 110 is equal to τⱼ₍ₜₓ₎+τ₂₍ᵣₓ₎. A delay time for the element 310 among the receive elements of the transducer 110 is equal to τⱼ₍ₜₓ₎+τ₃₍ᵣₓ₎, and a time delay for the element 320 among the receive elements of the transducer 110 is equal to τⱼ₍ₜₓ₎+τ₄₍ᵣₓ₎. When the element 330 among the receive elements of the transducer 110 is an i-th element, a delay time for the element 330 is equal to τⱼ₍ₜₓ₎+τᵢ₍ᵣₓ₎.

Thereafter, in response to the ultrasound transmitted by the single element 316 among the plurality of selected transmit elements from the elements of the transducer 110, the ultrasound medical apparatus 100 generates reception signals with time delays applied, which are defined concurrently corresponding to the paths through which the transmitted ultrasound is reflected at a receive focusing point (x, z) and arrives at the receive elements 310, 314, 316, 320, 330, and then combines the generated reception signals into a second combined signal.

The following describes a generation of a reception signal at the time of transmitting an ultrasound at the elements 310 and 316 according to some embodiments of the present disclosure with reference to FIG. 4A.

The ultrasound medical apparatus 100 concurrently combines the combined signal resulting from the ultrasound transmitted by the single element 310 among the plurality of selected transmit elements from the elements of the transducer 110 and the reflected ultrasound signals with time delays applied corresponding to the receive elements 310, 314, 316, 320 and 330 with the combined signal resulting from the ultrasound transmitted by the single element 316 among the plurality of selected transmit elements of the transducer 110 and the reflected ultrasound signals corresponding to the receive elements 310, 314, 316, 320 and 330, in order to generate a combined reception signal.

On the other hand, as shown in FIG. 4B, the ultrasound medical apparatus 100 may concurrently combine the first combined signal generated in response to the ultrasound transmitted by the single element 310 among a plurality of selected transmit elements from the elements of the transducer 110 by using reception signals with time delays applied corresponding to the paths through which the transmitted ultrasound is reflected at a receive focusing point (x, z) and arrives at the receive elements 310, 314, 316, 320, 330 with the second combined signal generated in response to the ultrasound transmitted by the single element 316 among the plurality of selected transmit elements of the transducer 110 by using reception signals with time delays applied corresponding to the paths through which the transmitted ultrasound is reflected at a receive focusing point (x, z) and arrives at the receive elements 310, 314, 316, 320, 330, so as to generate a third combined signal.

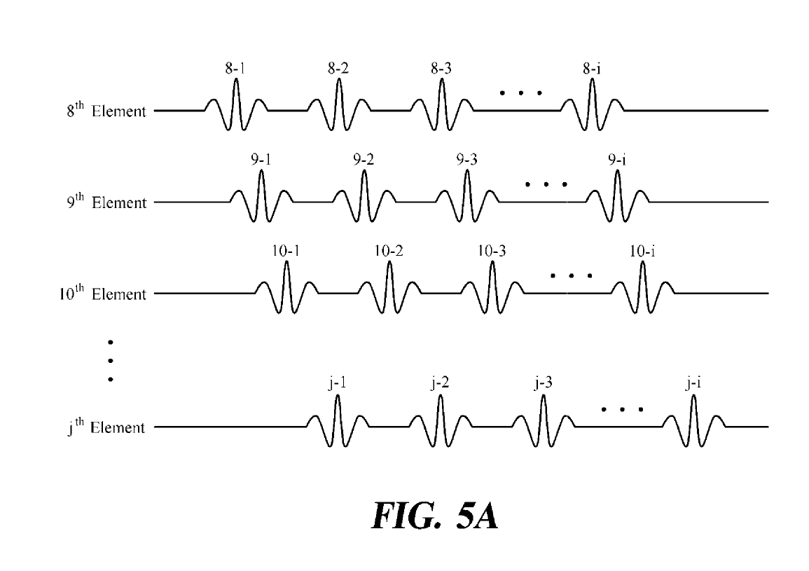

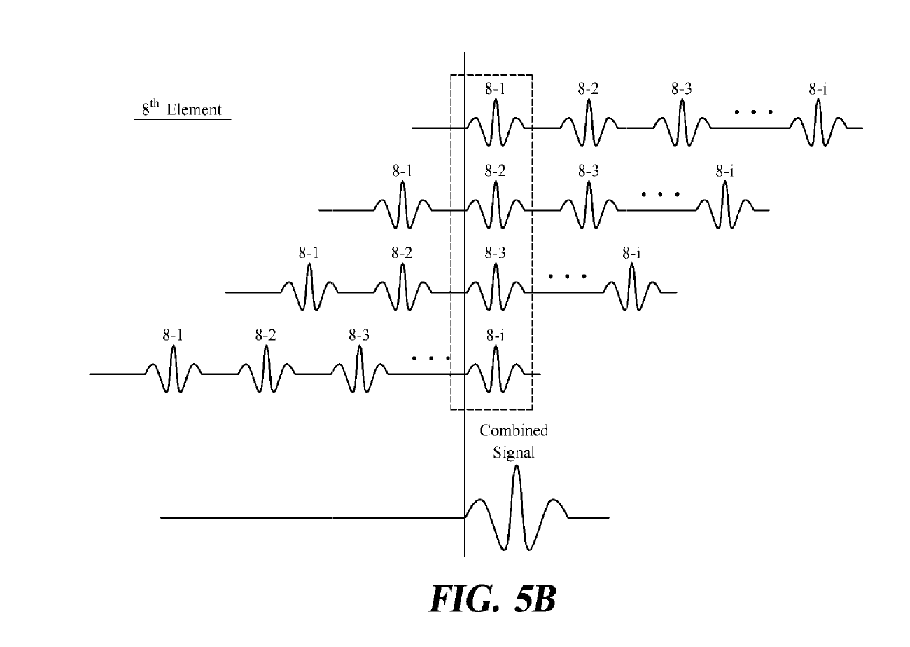

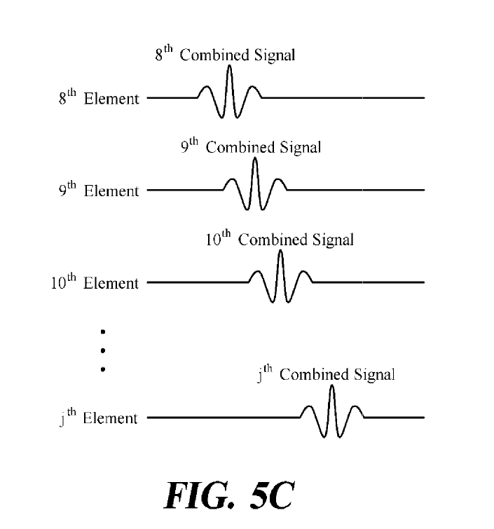

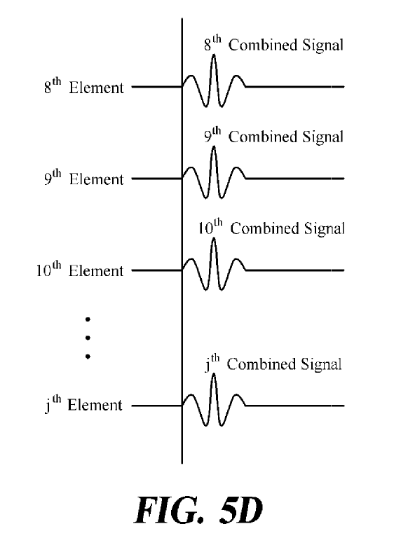

FIGS. 5A to 5D are schematic diagrams for illustrating a beamforming in a process of receiving a reflected signal according to some embodiments of the present disclosure.

In FIGS. 5A to 5D, the ultrasound medical apparatus 100 designates the plurality of transmit elements selected from the elements (all transmit elements) of the transducer 110 as, for example, 1st to j-th elements, and sets the receive elements to 1st to i-th elements.

As shown in FIG. 5A, for respective ones of a plurality of selected transmit elements (1st to j-th elements) from the elements of the transducer 110, the ultrasound medical apparatus 100 performs the beamforming taking account of time delays corresponding to the paths through which an ultrasound is transmitted from a single element among the plurality of selected transmit elements, reflected at a receive focusing point (x, z) in the field-of-view and arrives at respective receive elements (1st to j-th elements), by applying the time delays to reception signals at the respective receive elements.

The following describes a process, in which reflected signals corresponding to the ultrasounds transmitted from the 1st to j-th transmit elements selected from the elements of the transducer 110 are received with reference to 8th to i-th receive elements among the elements.

For example, when the 8th receive element among the receive elements is closest to the receive focusing point (x, z) in the field-of-view, the 8th receive element among the receive elements receives a reflected signal 8-1 corresponding to the ultrasound transmitted by the 8th transmit element among the elements selected from the transmit elements of the transducer 110, and then the 9th element next to the 8th element receives a reflected signal 9-1 corresponding to the ultrasound transmitted from the 8th transmit element among the elements selected from the transmit elements of the transducer 110. Thereafter, the 10th element next to the 9th element among the receive elements receives a reflected signal 10-1 corresponding to the ultrasound transmitted from the 8th transmit element among the elements selected from the transmit elements of the transducer 110, and the i-th receive element among the receive elements located next to the 10th element receives a reflected signal i-1 corresponding to the ultrasound transmitted from the 8th transmit element among the elements selected from the transmit elements of the transducer 110.

Further, the 8th receive element among the receive elements receives a reflected signal 8-2 corresponding to the 9th transmit element among the elements selected from the transmit elements of the transducer 110, and then the 9th element next to the 8th receive element among the receive elements receives a reflected signal 9-2 corresponding to the ultrasound transmitted from the 9th transmit element among the elements selected from the transmit elements of the transducer 110. Thereafter, the 10th element next to the 9th receive element among the receive elements receives a reflected signal 10-2 corresponding to the ultrasound transmitted from the 9th transmit element among the elements selected from the transmit elements of the transducer 110, and the i-th receive element among the receive elements located next to the 10th element receives a reflected signal i-2 corresponding to the ultrasound transmitted from the 9th transmit element among the elements selected from the transmit elements of the transducer 110.

Moreover, the 8th receive element among the receive elements receives a reflected signal 8-3 corresponding to the 10th transmit element among the elements selected from the transmit elements of the transducer 110, and then the 9th element next to the 8th receive element among the receive elements receives a reflected signal 9-3 corresponding to the ultrasound transmitted from the 10th transmit element among the elements selected from the transmit elements of the transducer 110. Thereafter, the 10th element next to the 9th receive element among the receive elements receives a reflected signal 10-3 corresponding to the ultrasound transmitted from the 10th transmit element among the elements selected from the transmit elements of the transducer 110, and the i-th element among the receive elements located next to the 10th element receives a reflected signal i-3 corresponding to the ultrasound transmitted from the 10th element among the elements selected from the transmit elements of the transducer 110.

Further, the 8th element among the receive elements receives a reflected signal 8-i corresponding to the j-th element among the elements selected from the transmit elements of the transducer 110, and then the 9th element next to the 8th element among the receive elements receives a reflected signal 9-i corresponding to the ultrasound transmitted from the j-th element among the elements selected from the transmit elements of the transducer 110. Thereafter, the 10th element next to the 9th element among the receive elements receives a reflected signal 10-i corresponding to the ultrasound transmitted from the j-th element among the elements selected from the transmit elements of the transducer 110, and the i-th element among the receive elements located next to the 10th element receives a reflected signal i-i corresponding to the ultrasound transmitted from the j-th element among the elements selected from the transmit elements of the transducer 110.

Thereafter, as shown in FIG. 5B, the ultrasound medical apparatus 100 generates a combined signal for the 8th element among the receive elements by applying a time delay based on the same time to each of the reflected signals (8-1, 8-2, 8-3, . . . , 8-i) corresponding to the ultrasounds transmitted from the 8th to j-th transmit element among the elements selected from the transmit elements of the transducer 110. Further, as shown in FIG. 5C, the ultrasound medical apparatus 100 generates a combined signal for the 9th element, a combined signal for the 10th element, . . . , and a combined signal for the j-th element by performing the above-mentioned process for each of the transmit elements (ninth to j-th elements) selected from the transmit elements of the transducer 110. Thereafter, as shown in FIG. 5D, the ultrasound medical apparatus 100 generates a reception signal by applying a concurrently defined time delay to the combined signals respectively corresponding to the 8th to j-th transmit elements among the elements selected from the transmit elements of the transducer 110 and combining the combined signals.

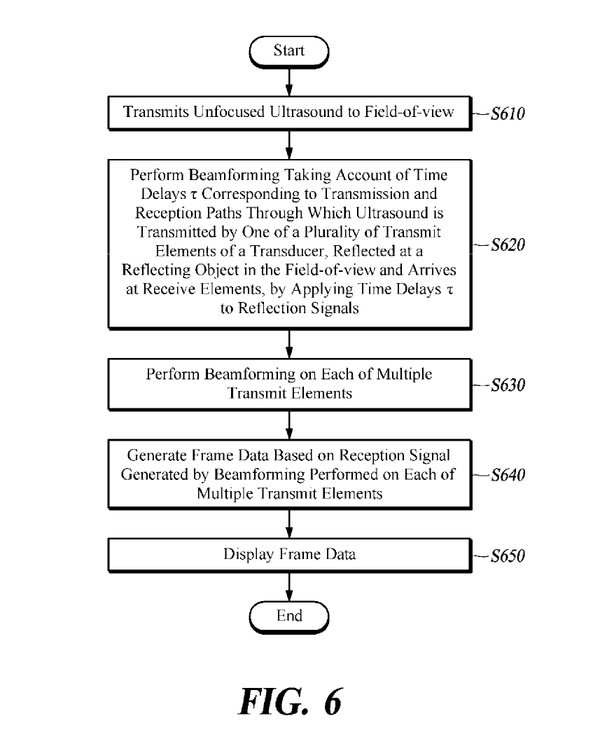

FIG. 6 is a flowchart of a process procedure for a method for performing a beamforming by using an unfocused ultrasound according to some embodiments of the present disclosure.

The ultrasound medical apparatus 100 transmits an unfocused ultrasound to the field-of-view by using the transducer (Step S610). Thereafter, the ultrasound medical apparatus 100 receives reflection signals corresponding to the unfocused ultrasound from the receive focusing point in the field-of-view by using the transducer 110.

For respective ones of a plurality of selected transmit elements from the elements of the transducer 110, the ultrasound medical apparatus 100 utilizes the beamformer 132 to perform a beamforming taking account of a transmission time delay τ corresponding to a transmission path through which an ultrasound is transmitted from a single element among the plurality of selected transmit elements and arrives at a receive focusing point in the field-of-view and reception time delays τ corresponding to reception paths through which ultrasound reflection signals reflected at the receive focusing point arrive at respective receive elements, by applying the transmission and reception time delays to the ultrasound reflection signals or reception signals at the respective receive elements (Step S620). In Step S620, the ultrasound medical apparatus 100 performs the beamforming with a transmission weight applied corresponding to the transmission path according to the depth of the receive focusing point in the field-of-view and a reception weight applied corresponding to the reception path.

The ultrasound medical apparatus 100 performs the beamforming on each of the elements selected from the transmit elements of the transducer 110 in a parallel manner (Step S630). In Step S630, the ultrasound medical apparatus 100 generates a combined signal by applying a time delay based on the same time to a plurality of reflected signals respectively corresponding to the ultrasounds transmitted from the elements selected from the transmit elements of the transducer 110 and combining the reflected signals, and generates a reception signal by applying a time delay based on the same time to the combined signals respectively corresponding to the elements selected from the transmit elements of the transducer 110 and combining the combined signals.

The ultrasound medical apparatus 100 generates frame data based on the reception signal generated by performing the beamforming on each of the elements selected from the transmit elements of the transducer 110 (Step S640). The ultrasound medical apparatus 100 allows the generated frame data to be displayed on a display unit (Step S650).

Although it is described that Steps S610 to S650 are sequentially performed in FIG. 6, the present disclosure is not limited to this scheme. The steps shown in FIG. 6 can also be performed by modifying one or more steps or by performing at a plurality of steps in parallel; and therefore, performing the steps shown in FIG. 6 is not limited to the chronological order.

The method for performing the beamforming by using an unfocused ultrasound according to some embodiments of the present disclosure shown in FIG. 6 can be implemented as a program and can be recorded in a computer-readable recording medium. The computer-readable recording medium that stores therein the program for implementing the method for performing the beamforming by using the unfocused ultrasound according to some embodiments of the present disclosure includes any type of recording device that stores therein computer-readable data.

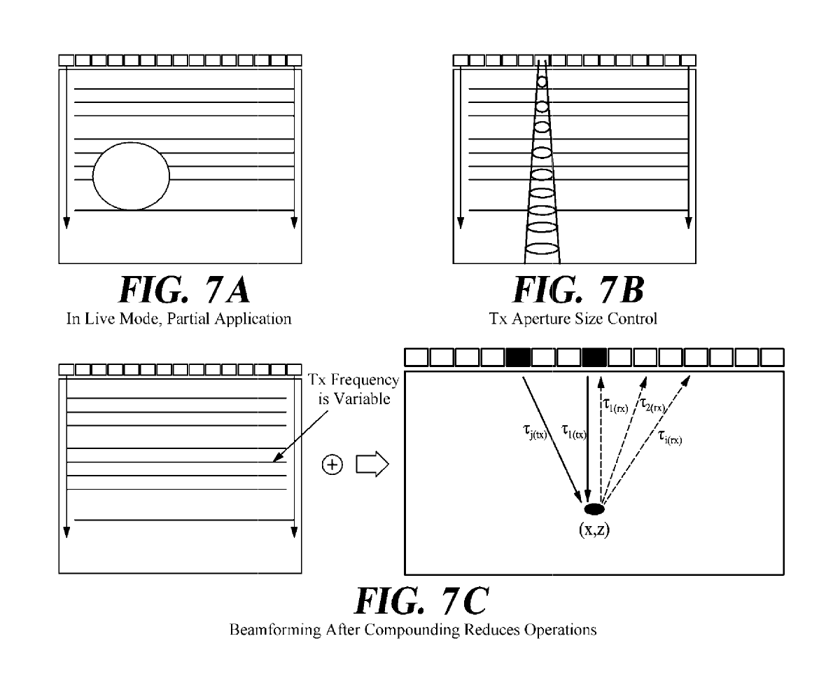

FIG. 7 is a schematic diagram for illustrating various beamforming processes according to some embodiments of the present disclosure.

As shown in FIG. 7A, the ultrasound medical apparatus 100 is configured to partially apply the beamforming according to some embodiments of the present disclosure to a live mode. Further, the ultrasound medical apparatus 100 is configured to partially apply the beamforming according to some embodiments of the present disclosure to a Doppler mode. Moreover, the ultrasound medical apparatus 100 is configured to partially apply the beamforming according to some embodiments of the present disclosure to a cine mode or fully apply the beamforming according to some embodiments of the present disclosure to the cine mode by selecting a specific track.

As shown in FIG. 7B, the ultrasound medical apparatus 100 is configured to change the number of elements selected from the transmit elements of the transducer 110 based on the depth of the receive focusing point in the field-of-view. When the number of elements selected from the transmit elements of the transducer 110 is changed, the number of receive elements of the transducer 110 is changed accordingly.

When the ultrasound medical apparatus 100 is configured to adjust the number of elements selected from the transmit elements of the transducer 110 based on the depth (location) of the receive focusing point in the field-of-view when transmitting the unfocused ultrasound to the field-of-view. Thereafter, the ultrasound medical apparatus 100 receives a reflected signal reflected at the receive focusing point. The ultrasound medical apparatus 100 applies a transmission weight corresponding to a transmission path through which the ultrasound arrives at the receive focusing point and a reception weight corresponding to a reception path through which the reflected signal arrives from the receive focusing point to each of the receive elements on the reflected signal based on the depth of the receive focusing point in the field-of-view, thus achieving an effect of a transmission dynamic focusing similar to an application of the apodization at the time of transmitting the unfocused ultrasound.

As shown in FIG. 7C, the ultrasound medical apparatus 100 allows unfocused ultrasounds having different frequencies from each other to be transmitted to the field-of-view, and generates frequency compound frame data of at least one frame data having different frequencies before performing the beamforming. Thereafter, the ultrasound medical apparatus 100 performs the beamforming on the frequency compound frame data.

Although exemplary embodiments of the present disclosure have been described for illustrative purposes, those skilled in the art will appreciate that various modifications, additions and substitutions are possible, without departing from the idea and scope of the claimed invention. Specific terms used in this disclosure and drawings are used for illustrative purposes and not to be considered as limitations of the present disclosure. Therefore, exemplary embodiments of the present disclosure have been described for the sake of brevity and clarity. Accordingly, one of ordinary skill would understand the scope of the claimed invention is not to be limited by the explicitly described above embodiments but by the claims and equivalents thereof.

### CROSS-REFERENCE TO RELATED APPLICATION

This application claims priority under 35 U.S.C. §119(a) of Patent Application No. 10-2013-0146925, filed on Nov. 29, 2013 in Korea, the entire content of which is incorporated herein by reference. In addition, this non-provisional application claims priority in countries, other than the U.S., with the same reason based on the Korean patent application, the entire content of which is hereby incorporated by reference.

1. A method for performing a beamforming by an ultrasound medical apparatus, the method comprising:
transmitting, by a transducer, an unfocused ultrasound to a field-of-view;
calculating a transmission delay time of a transmission path through which an ultrasound transmitted by one of a plurality of transmit elements of the transducer arrives at a receive focusing point and calculating reception delay times of reception paths through which the ultrasound reflected at the receive focusing point arrives at a plurality of receive elements respectively;
generating a plurality of delay signals for respective reception signals of the receive elements by applying the calculating of the transmission delay time and the reception delay times to remaining transmit elements of the plurality of transmit elements; and
performing the beamforming by summing the plurality of delay signals.

2. The method according to claim 1, wherein the transmit elements are determined based on a depth of the receive focusing point.

3. The method according to claim 1, wherein a number of the transmit elements increases as a depth of the receive focusing point increases.

4. The method according to claim 1, wherein the performing of the beamforming by summing comprises:
determining weights for respective ones of the transmit elements based on the receive focusing point and positions of the transmit elements;
generating weighted delay signals by applying the weights to the plurality of delay signals; and
performing the beamforming by weighted summing the weighted delay signals having been generated.

5. The method according to claim 1, wherein the performing of the beamforming by summing comprises:
determining first weights for respective ones of the transmit elements based on the receive focusing point and positions of the transmit elements;
determining second weights for respective ones of the receive elements based on the receive focusing point and positions of the receive elements;
generating weighted delay signals by applying the first weights and the second weights to the plurality of delay signals; and
performing the beamforming by weighted summing the weighted delay signals having been generated.

6. The method according to claim 1, wherein the receive focusing point comprises a point selected in a region-of-Interest.

7. The method according to claim 1, wherein, in a multi-gate Doppler mode, the receive focusing point comprises a gate position.

8. The method according to claim 1, further comprising:
storing one or more reflection signals reflected at the field-of-view corresponding to the unfocused ultrasound; and
causing, when an image reconstruction mode is selected by a user instruction, the generating of the plurality of delay signals and the performing of the beamforming by summing to proceed by using the reflected signals stored at the storing.

9. An ultrasound medical apparatus, comprising:
a transducer configured to transmit unfocused ultrasounds to a field-of-view; and
a beamformer configured to
calculate a transmission delay time of a transmission path through which an ultrasound transmitted by one of a plurality of transmit elements of the transducer arrives at a receive focusing point and calculate reception delay times of reception paths through which the ultrasound reflected at the receive focusing point arrives at a plurality of receive elements, respectively,
generate a plurality of delay signals for respective reception signals of the receive elements by applying calculations of the transmission delay time and the reception delay times to remaining transmit elements of the plurality of transmit elements, respectively, and
perform a beamforming by summing the plurality of delay signals.

10. The ultrasound medical apparatus according to claim 9, wherein the beamformer is configured to
determine weights for respective ones of the transmit elements based on the receive focusing point and positions of the transmit elements,
generate weighted delay signals by applying the weights to the plurality of delay signals, and
perform the beamforming by weighted summing the weighted delay signals.

11. The ultrasound medical apparatus according to claim 9, wherein the beamformer is configured to
determine first weights for respective ones of the transmit elements based on the receive focusing point and positions of the transmit elements,
determine second weights for respective ones of the receive elements based on the receive focusing point and positions of the receive elements,
generate weighted delay signals by applying the first weights and the second weights to the plurality of delay signals, and
perform the beamforming by weighted summing the weighted delay signals.

12. The ultrasound medical apparatus according to claim 9, wherein the beamformer is configured to
cause the unfocused ultrasounds having different frequencies from each other to be transmitted to the field-of-view,
generate at least one frame data based on reflection signals corresponding to the unfocused ultrasounds having different frequencies from each other before performing the beamforming,
generate frequency compound frame data by frequency compounding the at least one frame data, and
perform the beamforming on the frequency compound frame data.

13. The ultrasound medical apparatus according to claim 9, wherein the unfocused ultrasounds comprise at least one of plane waves or wide beams.
